(19) United States
(12) Patent Application Publication
(10) Pub. No.: US 2021/0106260 A1
(43) Pub. Date: Apr. 15, 2021

(54) MEDIATOR-FREE BIOCHEMICAL SENSING DEVICE AND METHOD FOR NONINVASIVELY AND ELECTROCHEMICALLY SENSING IN VIVO BIOCHEMICALS

(71) Applicant: THE REGENTS OF THE UNIVERSITY OF CALIFORNIA, Oakland, CA (US)

(72) Inventors: Sam Emaminejad, Los Angeles, CA (US); Yichao Zhao, Los Angeles, CA (US); Bo Wang, Los Angeles, CA (US); Xuanbing Cheng, Los Angeles, CA (US)

(73) Assignee: THE REGENTS OF THE UNIVERSITY OF CALIFORNIA, Oakland, CA (US)

(21) Appl. No.: 17/067,338

(22) Filed: Oct. 9, 2020

Related U.S. Application Data
(60) Provisional application No. 62/914,024, filed on Oct. 11, 2019.

(51) Int. Cl. A61B 5/145; A61B 5/1477; A61B 5/1486; C25D 7/00; C25D 3/50; C25D 15/00

(52) U.S. Cl. CPC A61B 5/14532; A61B 5/14546; A61B 5/1477; C25D 15/00; C25D 7/00; C25D 3/50; A61B 5/1486

(57) ABSTRACT

Example implementations include a method of manufacturing a biochemical sensor by forming a fluid region in a microfluidic layer, forming a reference electrode on a planar surface of an electrode layer, forming a biochemical sensor electrode on the planar surface, forming a selective membrane on the biochemical sensor electrode, forming an enzymatic material including a biochemical sensing material on the selective membrane, and bonding the electrode layer to the microfluidic layer. Example implementations also include a device with a reference electrode disposed on a planar surface of an electrode layer, a biochemical sensor electrode disposed on the planar surface, a selective membrane disposed on the biochemical sensor electrode and impermeable to at least one biochemical interferent, and an enzymatic layer disposed on the selective membrane and electrically responsive to a biochemical.

### CROSS-REFERENCE TO RELATED PATENT APPLICATIONS

This application claims priority to U.S. Provisional Patent Application Ser. No. 62/914,024, entitled “Mediator-Free Electroenzymatic Sensing with Enchanced Sensitivity and Selectivity for Wearable Metabolite and Nutrient Monitoring Applications,” filed Oct. 11, 2019, the contents of such application being hereby incorporated by reference in its entirety and for all purposes as if completely and fully set forth herein.

### FEDERALLY SPONSORED RESEARCH OR DEVELOPMENT

This invention was made with government support under Grant Number 1722972, awarded by the National Science Foundation. The government has certain rights in the invention.

### TECHNICAL FIELD

The present implementations relate generally to biochemical sensing, and more particularly to a mediator-free biochemical sensing device and noninvasively and electrochemically sensing in vivo biochemicals.

### BACKGROUND

Health monitoring is increasingly desired to perform increasingly accurate health diagnostics and guide improved health outcomes for increasing numbers of users and activity scenarios. In particular, detection of biochemical levels of biofluids secreted by a user can provide significant health data and, in turn, drive significantly improved health outcomes. However, conventional systems may not effectively detect and isolate biochemicals in biofluids at in vivo sites noninvasively and accurately. In addition, conventional systems may detect and isolate biochemicals using a biochemical mediator subject to degradation through chemical interaction with target analyte during sensing. Thus, a technological solution for a mediator-free biochemical sensing device for noninvasively and electrochemically sensing in vivo biochemicals is desired.

### SUMMARY

Example implementations include a method of manufacturing a biochemical sensor by forming a fluid region in a microfluidic layer, forming a reference electrode on a planar surface of an electrode layer, forming a biochemical sensor electrode on the planar surface, forming a selective membrane on the biochemical sensor electrode, forming an enzymatic material including a biochemical sensing material on the selective membrane, and bonding the electrode layer to the microfluidic layer.

Example implementations also include a device with a reference electrode disposed on a planar surface of an electrode layer, a biochemical sensor electrode disposed on the planar surface, a selective membrane disposed on the biochemical sensor electrode and impermeable to at least one biochemical interferent, and an enzymatic layer disposed on the selective membrane and electrically responsive to a biochemical.

Example implementations also include a method of electrically detecting a biochemical by contacting a biochemical sensor electrode to a biological surface, obtaining a biofluid at the biochemical sensor electrode from the biological surface, filtering an interferent at a selective membrane disposed between the biochemical sensor electrode and the biological surface, obtaining a response current associated with the biofluid at the biochemical sensor electrode, and generating a quantitative biochemical response based at least partially on the response current.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates an example device in accordance with present implementations.

FIG. 1B illustrates a further example device in accordance with present implementations.

FIG. 2 illustrates a cross-sectional view of an example electrochemical sensor further to the example devices of FIGS. 1A and 1B.

FIG. 3 illustrates an example electronic sensor device in accordance with present implementations.

FIG. 4A illustrates an example method of manufacturing an example electrochemical sensor in accordance with present implementations.

FIG. 4B illustrates an example method of manufacturing an example electrochemical sensor further to the example method of FIG. 4A.

FIG. 5A illustrates an example method of electrically sensing a biochemical in accordance with present implementations.

FIG. 5B illustrates an example method of electrically sensing a biochemical further to the example method of FIG. 5A.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Wearable biomarker sensors present great potential for transforming personalized healthcare and precision medicine, because they allow frequent and convenient harvesting of relevant physiological data from non-invasively accessible biofluid samples such as sweat and saliva, which share biomarker partitioning pathways with blood. In some implementations, by integrating these devices within distributed computing systems, with computer networks, Internet-of-Things (IoT) infrastructure, or the like, users can create personal health and wellness databases that grant them more autonomy in monitoring their physiological well-being. In some implementations, aggregated information provides actionable feedback to the user with respect to adopting/modifying lifestyle routines and daily activities such as nutrition and physical exercise. To this end, tracking circulating metabolites, including but not limited to glucose and lactate, and nutrients, including but not limited to choline, plays a significant role in rendering a complete view of the body's dynamic chemistry. To measure these molecules in target biofluids in a wearable format, electrochemical sensing interfaces in accordance with present implementations are especially suitable. Advantages of present implementations include, but are not limited to, simplicity of manufacturing, low cost of materials and manufacturing, and ease of integration with readout electronics to realize sample-to-answer sensing systems. As one example, the use of Prussian Blue can increase susceptibility of mediator-based sensor response to dynamic concentrations of ionic species. Further, in this example, the use of Prussian Blue can result in a loss of electrocatalytic sensor activity due to degradation of the Prussian Blue's framework.

Thus, it is advantageous to provide a biochemical sensor to provide a noninvasive electrochemical sensor effectively operable in a wide variety of use cases. It is further advantageous to provide a biochemical sensor in a configuration including a plurality of stacked layers to minimize torque, stress, shear force, and the like applied to the sensor in response to movement, pressure, acceleration or the like by a user conducting an activity while wearing the biochemical sensor array. It is further advantageous to provide a biochemical sensor in a configuration including a plurality of flexible layers to minimize torque, stress, shear force, and the like applied to the sensor in response to movement, pressure, acceleration or the like by a user conducting an activity while wearing the biochemical sensor array. As one example, a user wearing a biochemical sensor on a limb may apply significant forces to the biochemical sensor thereon during the course of strenuous activity in which biofluid is released. As one example, strenuous activity can include sweat-inducing exercise, exertion, and the like. As another example, strenuous activity can include running, boxing, cycling, and the like. Thus, advantages of present implementations include, but are not limited to, superior sensitivity, selectivity and stability in a sensing interface.

It is advantageous to detect glucose, lactate, and choline, because health conditions associated with excessive accumulation or deficiency can be detected therethrough and reversed by active interventions within a short period assuming timely notification of onset. For example, sweat glucose can track blood glucose levels. In this example, feedback from noninvasive wearable sensors indicating abnormally high or low levels can motivate insulin administration treatment or food intake respectively. Similarly, sweat lactate is a possible indicator of physical stress and aerobic/anaerobic transitions. As one example, tracking its presence in sweat during exercise cam determine when breaks are required. Finally, choline is important for general organ function and serves as a precursor to the neurotransmitter acetylcholine. As one example, short-term choline deficiency, which can cause fat accumulation in the liver and other effects, can be reversed within a few days by reintroducing choline to the individual's diet.

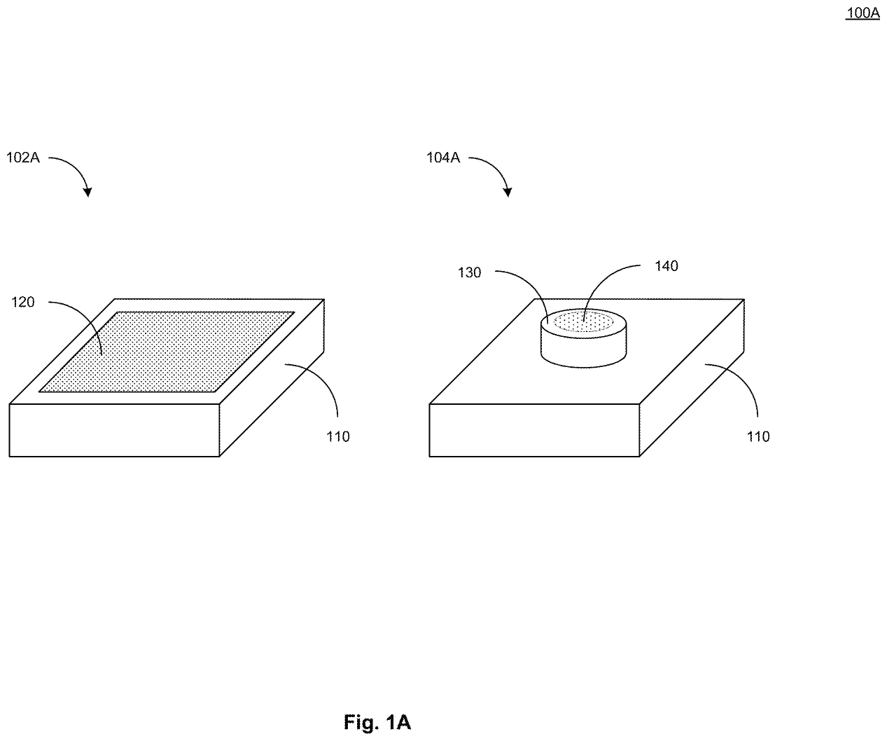

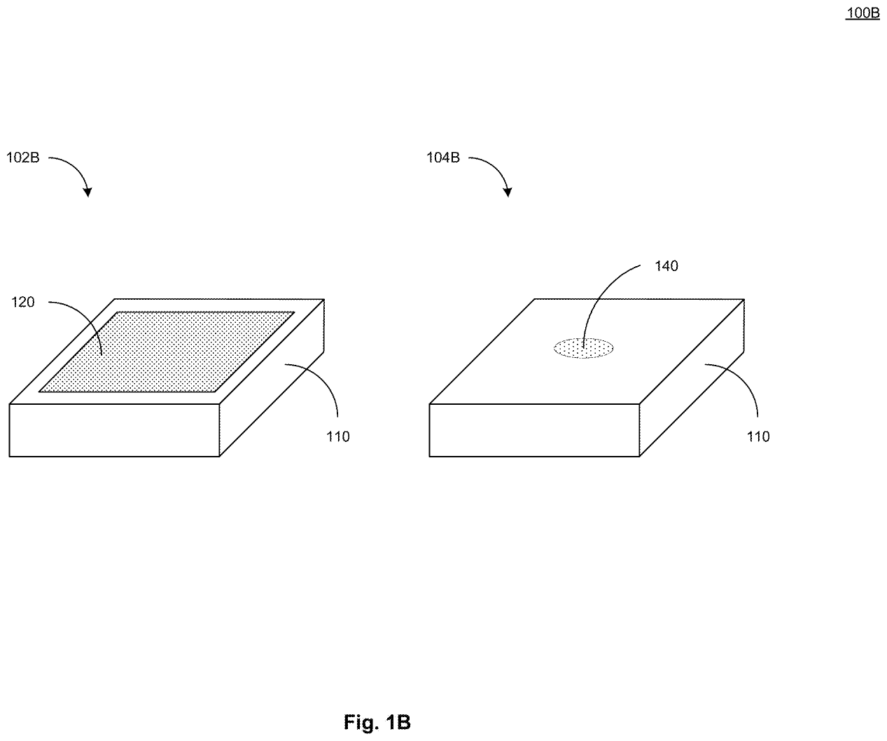

FIG. 1A illustrates a top view 102A and a bottom view 104A of an example device 100A in accordance with present implementations. As illustrated by way of example in FIG. 1, the example device 100A includes a housing 110, a display device 120, a stimulation module 130, and an electrochemical sensor 140.

The housing 110 contains or the like one or more sensors, electrical devices, electronic devices, mechanical structures, and the like. In some implementations, the housing 110 includes a plastic material, a polymer material, electrically insulating material, waterproof material, water resistant material, or the like. In some implementations, the housing 110 includes a 3D-printed structure. In some implementations, the housing 110 includes a first face oriented or orientable toward a biological surface. In some implementations, the housing 110 includes a second face oriented or orientable away from the biological surface. In some implementations, the first face and the second face of the housing 110 are disposed on opposite surfaces of the housing 110.

The display device 120 is operable to display one or more biochemical characteristics associated with a biofluid. In some implementations, the biofluid includes one or more characteristics associated with a biochemical therein. In some implementations, the biofluid includes one or more of glucose, choline, and lactate. In some implementations, the characteristics include a pH characteristic. In some implementations, the display device 120 includes an electronic display. In some implementations, the electronic display includes a liquid crystal display (LCD), a light-emitting diode (LED) display, an organic light-emitting diode (OLED) display, or the like. In some implementations, the display device 120 is housed at least partially within the housing 120, on its second face oriented or orientable away from the biological surface.

The stimulation module 130 is operable to apply electrical energy to the biological surface according to one or more electrical output patterns. In some implementations, the stimulation module 130 is operable to apply electrical energy to the biological surface in accordance with an iontophoresis process. In some implementations, the stimulation module 130 is operable to induce a biological reaction from the biological surface. In some implementations, a biological reaction includes release of biofluid from the biological surface. As one example, the stimulation module 130 can apply electrical energy to skin to induce release of sweat. In some implementations, the stimulation module 130 includes one or more electrical, electronic, and logical devices. In some implementations, the stimulation module 130 includes one or more integrated circuits, transistors, transistor arrays, or the like.

The electrochemical sensor 140 is operable to detect one or more biochemicals in contact therewith or contactable therewith. In some implementations, the electrochemical sensor 140 is operable to detect a plurality of biochemical. In some implementations, the electrochemical sensor 140 includes one or more electrode with biochemically-sensitive electrode terminals. In some implementations, the electrochemical sensor 140 includes a plurality of electrodes arranged in a geometric pattern. As one example, the plurality of electrodes can be arranged in a grid pattern including an arbitrary number of electrodes in a length direction and a width direction perpendicular to the length direction. In some implementations, the electrochemical sensor 140 include at least one opening, chamber, or the like, to receive biofluid from the biological surface and to contactably couple the biofluid to at least one electrode terminal, biochemically-sensitive electrode terminal, or a combination thereof. In some implementations, the biochemical sensor 140 includes one or more polymers, plastics, or the like. In some implementations, the biochemical sensor 140 includes one or more films, sheets, layers, or the like. In some implementations, the biochemical sensor 140 is or includes one or more films, sheets, layers, or the like arranged in a planar structure. In some implementations, the biochemical sensor 140 is or includes a flexible structure deformable, bendable, or the like in one or more planar directions.

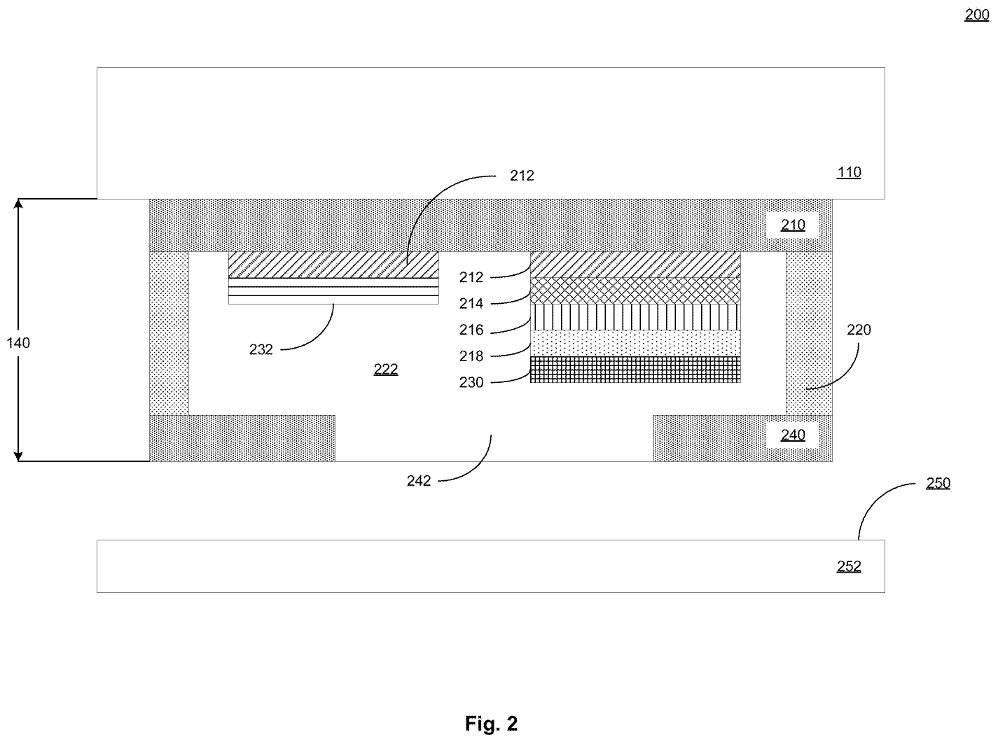

FIG. 1B illustrates a top view 102B and a bottom view 104B of a further example device 100B in accordance with present implementations. As illustrated by way of example in FIG. 2, the example device 100B includes the housing 110, the display device 120, and the electrochemical sensor 140. In some implementations, the example device 100B includes the housing 110 and the display device 120 correspondingly to those of the example device 100A. In some implementations, the example device 100B does not include the stimulation module 130, and the electrochemical sensor 140 is disposed on, over, in, or the like, the housing 110.

FIG. 2 illustrates a cross-sectional view 200 of an example electrochemical sensor 140 further to the example devices of FIGS. 1A and 1B. As illustrated by way of example in FIG. 2, an example electrochemical sensor 140 includes an electrode layer 210, a biochemical sensor electrode, a reference electrode, a microfluidic layer 220, a fluid chamber 222, and a barrier layer 240. In some implementations, the biochemical sensor electrode includes a separate base electrode layer 212, a carbon nanotube layer 214, a platinum layer 216, a selective membrane 218, and an enzymatic material 230. In some implementations, the reference electrode includes a base electrode layer 212 and a reference electrode cap layer 232. In some implementations, one or more layers of one or more of the biochemical sensor electrode and the reference electrode are partially or fully intermingled, combined, or the like as a result of a fabrication process in which one or more layers are sequentially disposed to form the electrodes.

In some implementations, the electrochemical sensor 140 is coupled to, integrated with, integrable with, or the like, the housing 110 and any components therein. In some implementations, the electrochemical sensor 140 is indirectly coupled to the housing 110 by the stimulation module. Alternatively, in some implementations, the electrochemical sensor 140 is directly coupled to the housing 110, where the housing includes the stimulation module 130. Alternatively, in some implementations, the electrochemical sensor 140 is directly coupled to the housing 110, where the housing does not include the stimulation module 130. In some implementations, the electrochemical sensor is contactable with a biological surface 250 of a biological object 252.

The electrode layer 210 includes a planar surface having at least one electrode formed thereon. In some implementations, the electrode layer 210 includes an adhesive conductive film bonding or operable to bond the electrode layer 210 to the housing 110. In some implementations, the electrode layer 210 has a first planar surface having one or more electrodes patterned, deposited, or the like, thereon. In some implementations, the electrode layer 210 includes a second surface opposite to the first planar surface and in direct or indirect contact with the housing 110. Alternatively, in some implementations, the electrode layer 210 includes a second surface opposite to the first planar surface and in direct or indirect contact with the stimulation module 130. In some implementations, the electrode layer 210 is or includes a polyethylene terephthalate (PET) film. In some implementations, the electrode layer 210 is operatively coupled to one or more electrical, electronic, or like components housed at least partially within the housing 110. In some implementations, the electrode layer 210 has a thickness of approximately 100 micrometers in a depth direction perpendicular to a plane thereof.

In some implementations, the electrode layer 210 is directly operatively coupled to one or more of the electrical, electronic, or like components of the housing 110 by a conductive characteristic thereof. As one example, an electrical conductivity characteristic of the electrode layer 210 can permit transmission of electrical current, voltage, signal, or the like between an electrode disposed on a first surface of the electrode layer 210 and an electrical or electronic component in contact with a second opposite surface of the electrode layer 210. In some implementations, individual electrodes of a plurality fabricated on the electrode layer 210 are disposed at a predetermined distance from each other to minimize or eliminate electrical interference from differing electrical current, voltage, signal, or the like traveling perpendicularly through the plane of the electrode layer 210 and associated with different electrodes disposed on the electrode layer 210.

The base electrode layer 212 includes at least one metallic portion forming at least part of at least one electrode terminal, biochemically-sensitive electrode terminal, or a combination thereof. In some implementations, the electrode material 212 is disposed on a first planar surface of the electrode layer 210 in one or more electrically isolated, electrically disconnected, or like configurations. In some implementations, the base electrode layer 212 is disposed in a grid pattern on the electrode layer 210. In some implementations, the base electrode layer 212 is or includes one or more noble metal electrode films. As one example, the base electrode layer 212 can include gold (Au) or the like, or a combination thereof. As another example, the base electrode layer 212 can include nanoparticles including gold (Au) or the like, or a combination thereof. In some implementations, the base electrode layer 212 is in direct contact with the electrode layer 210. In some implementations, at least one of the biochemical sensor electrode and the reference electrode includes a noble metal electrode material disposed on a metallic adhesive layer. As one example, a metallic adhesive layer can include one or more of chromium (Cr) or titanium (Ti). Thus, in some implementations, the base electrode layer 212 is disposed on a chromium layer or the like, and the chromium layer or the like is disposed directly on the electrode layer 210 to increase conductivity through the electrode layer 210. In some implementations, the base electrode layer 212 of one or more of the biochemical sensor electrode and the reference electrode have substantially circular shape and a diameter of 4 mm. In some implementations, the based electrode layer has a thickness of 100 nm Au. In some implementations, a Cr layer adjacent to the base electrode layer 212 has a thickness of 30 nm.

The carbon nanotube layer 214 is disposed adjacent to the base electrode layer 212 on the biochemical sensor electrode. In some implementations, the carbon nanotube layer 214 is or includes a multi-walled carbon nanotube (MWCNT) layer.

The platinum layer 216 is disposed adjacent to the carbon nanotube layer 214 on the biochemical sensor electrode. As one example, the base electrode layer 212 can include gold (Au), platinum (Pt), or the like, or a combination thereof. As another example, the base electrode layer 212 can include nanoparticles including gold (Au), platinum (Pt), or the like, or a combination thereof. It is advantageous to use a platinum (Pt)-based electrode, which is naturally inert against ionic species, unlike various mediator substances, including PB. Thus, in some implementations, Pt nanoparticles (PtNP) are electrodeposited onto the carbon nanotube layer 214 to achieve superior electrocatalytic capability toward the detection of hydrogen peroxide. In some implementations, MWCNT and PtNP together transduce chemical signals to electrical signals that correspond to the target analyte information as a process corresponding to the electroanalysis of hydrogen peroxide. Thus, in some implementations, the hybridization of MWCNT and PtNP results in an active electrode surface with high sensitivity for detection of H₂O₂.

The selective membrane 218 is disposed adjacent to the platinum layer 216 on the biochemical sensor electrode. In some implementations, the selective membrane is or includes a permselective layer. In some implementations, the selective membrane is or includes poly-m-phenylenediamine, PPD 12-13 (PPD), which only allows small and neutral molecules (e.g., H₂O₂) to pass through to the electroanalysis layer. In some implementations, the permselective layer is a diffusion limiting layer that inhibits H₂O₂ detection sensitivity to 44±15 μA/mM/cm2.1.) In some implementations, the selective membrane 218 rejects one or more electroactive interferents, including but not limited to uric acid (UA) and the like, while allowing H₂O₂ to penetrate and reach the underlying electroanalysis layers 212, 214 and 216.

The biochemical sensor material 214 includes at least one sensor responsive to at least one chemical, biochemical, or the like. In some implementations, the biochemical sensor material 214 is disposed on a first planar surface of the electrode layer 210 in one or more electrically isolated, electrically disconnected, or like configurations. In some implementations, the biochemical sensor material 214 is disposed on the electrode layer 210. In some implementations, the biochemical sensor material 214 includes glucose oxidase and is electrically responsive to contact with glucose. In some implementations, the biochemical sensor material 214 includes choline oxidase and is electrically responsive to contact with choline. In some implementations, the biochemical sensor material 214 includes lactate oxidase and is electrically responsive to contact with lactate. In some implementations, sensing interfaces measure one or more of glucose, lactate, and choline, as example critical metabolites and nutrients accessible in biofluids such as sweat and saliva.

The microfluidic layer 220 includes at least one cavity in at least one structure operable to capture fluid. In some implementations, the cavity of the microfluidic structure 220 includes at least one opening in a planar structure thereof. In some implementations, the microfluidic layer 220 is or includes a flexible planar structure. In some implementations, the microfluidic layer 220 has a thickness of 170 micrometers. As one example, the microfluidic structure can be a flexible plastic film. As another example, the microfluidic structure can be a flexible plastic adhesive tape. In some implementations, the microfluidic layer 220 is an adhesive tape layer disposed between the electrode layer 210 and the barrier layer 240. In some implementations, the adhesive layer is or includes a double-sided adhesive tape layer. In some implementations, the microfluidic layer 220 is bonded to the first surface of the electrode layer 210 including at least one of the base electrode layers 212 of the biochemical sensor electrode and the reference electrode.

In some implementations, the microfluidic layer 220 includes at least one fluid chamber 222. In some implementations, the cavity of the microfluidic structure 220 includes at least one fluid chamber 222 formed in a planar structure thereof. In some implementations, the fluid chamber 222 is one of a plurality of fluid chambers disposed within the microfluidic layer 220. In some implementations, the fluid chamber 222 is one of a plurality of fluid chambers disposed in a grid pattern correspondingly to a grid pattern of the electrode material 212 on the electrode layer 210. In some implementations, the fluid chamber 222 is aligned with at least one electrode including at least the electrode material 212. Thus, in some implementations, the fluid chamber at least partially encloses an electrode including at least the electrode material 212 and disposed on the electrode layer 210. In some implementations, the microfluidic layer 220 includes at least one fluid channel 224. In some implementations, the cavity of the microfluidic structure 220 includes at least one fluid channel 224 formed in a planar structure thereof. In some implementations, the fluid channel 224 is one of a plurality of fluid channels disposed within the microfluidic layer 220. In some implementations, the fluid channel 224 is one of a plurality of fluid channels connecting a plurality of fluid chambers disposed in a grid pattern correspondingly to a grid pattern of the electrode material 212 on the electrode layer 210.

The enzymatic material 230 is disposed adjacent to the selective membrane 218 of the biochemical sensor electrode. In some implementations, that enzymatic material includes a stabilizer component and a biochemically responsive component. In some implementations, the biochemically responsive component is or includes an oxidase. In some implementations, the biochemically responsive component is or includes at least one of glucose oxidase, lactate oxidase, and choline oxidase. The reference electrode cap layer 232 is disposed adjacent to the base electrode layer 212 of the reference electrode. In some implementations, the reference electrode cap layer is or includes silver chloride (AgCl).

The barrier layer 240 includes a planar structure bonded to the microfluidic layer 220. In some implementations, the barrier layer 240 has a first planar surface having one or more cavities, openings, or the like 242 formed therein. In some implementations, the barrier layer 240 is bonded to the microfluidic structure 220. In some implementations, the cavities, openings or the like, of the barrier layer 240 allow fluid to enter the fluid chamber 222 from a biological surface. In some implementations, the barrier layer 240 includes a second surface opposite to the first planar surface and in direct or indirect contact with the biological surface. Alternatively, in some implementations, the barrier layer 240 includes a second surface opposite to the first planar surface. In some implementations, the barrier layer 240 is or includes a biocompatible film, biocompatible adhesive film, or the like. Thus, in some implementations, microfluidic channels are chambers are cavities bounded in a direction by the electrode layer 210, bounded on one or more sides by the microfluidic layer 220, and bounded below the barrier layer 240 as a floor.

The biological surface 250 is or includes a surface of living tissue, biological matter, or the like. In some implementations, the biological surface 250 includes partially or fully exposed skin or the like of a human, animal, plant, or the like. In some implementations, the biological surface secretes or is capable of secreting one or more fluid having one or more biochemicals therein. In some implementations, biochemical include, but are not limited to, glucose, lactate, choline, and the like. The biological object 252 includes the biological surface 250 and is or includes living tissue, biological matter, or the like. In some implementations, the biological object 252 is or includes human skin, animal skin, and the like. In some implementations, the biological object 252 includes, directs or is responsive to secrete one or more biofluids. An one example, the biological object 252 can be responsive to electrical stimulation to induce secretion of sweat by an iontophoresis process or the like.

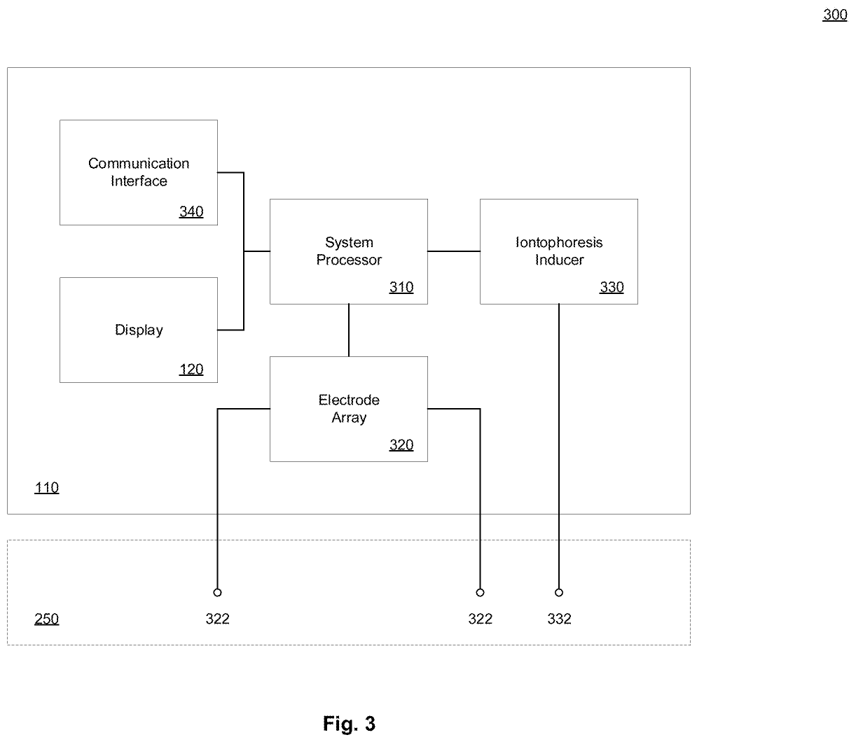

FIG. 3 illustrates an example electronic sensor device in accordance with present implementations. As illustrated by way of example in FIG. 3, an example electronic sensor device 300 includes a system processor 310, an electrode array 320, a biosensor electrode terminal 322, an iontophoresis inducer 330, an iontophoresis electrode terminal 332, a communication interface 340, and the display device 120. In some implementations, the example electronic sensor device 300 is housed at least partially within the housing 110. In some implementations, the electronic sensor device 300, including but not limited to the biosensor electrode terminal 322 and the iontophoresis electrode terminal 332, is contactable with a biological surface 250.

The system processor 310 is operable to execute one or more instructions associated with input from the electrochemical sensor 140. In some implementations, the system processor 310 is an electronic processor, an integrated circuit, or the like including one or more of digital logic, analog logic, digital sensors, analog sensors, communication buses, volatile memory, nonvolatile memory, and the like. In some implementations, the system processor 310 includes but is not limited to, at least one microcontroller unit (MCU), microprocessor unit (MPU), central processing unit (CPU), graphics processing unit (GPU), physics processing unit (PPU), embedded controller (EC), or the like. In some implementations, the system processor 310 includes a memory operable to store or storing one or more instructions for operating components of the system processor 310 and operating components operably coupled to the system processor 310. In some implementations, the one or more instructions include at least one of firmware, software, hardware, operating systems, embedded operating systems, and the like. It is to be understood that the system processor 310 or the device 300 generally can include at least one communication bus controller to effect communication between the system processor 310 and the other elements of the device 300.

The electrode array 320 is operable to detect electrical responses from one or more of the biosensor electrode terminals 322 and to communicate the electrical responses to the system processor. In some implementations, the electrode array 320 includes a power source, battery, power controller, potentiostat, or the like, operable to apply or maintain a working voltage at one or more of the biosensor electrode terminals 322. In some implementations, the working voltage is a voltage of +0.5 V. In some implementations, the electrode array 320 includes one or more analog signal processors, transformers, or the like, operable to convert one or more received response currents from one or more of the biosensor electrode terminals 332 to one or more corresponding response voltages or the like. As one example, the electrode array 320 can convert a response current associated with a particular biochemical response to a response voltage having a magnitude corresponding to the biochemical response. In some implementations, the electrode includes a low pass filter or the like operable to minimize motion-inducted noise in the electrical response current received from one or more of the biosensor electrode terminals. In some implementations, the low pass filter operates at or approximately at 1 Hz.

In some implementations, the electrode array 320 includes one or more logical or electronic devices including but not limited to integrated circuits, logic gates, flip flops, gate arrays, programmable gate arrays, and the like. In some implementations, the electrode array 320 includes the electrochemical sensor 140. It is to be understood that any electrical, electronic, or like devices, or components associated with the electrode can also be associated with, integrated with, integrable with, replaced by, supplemented by, complemented by, or the like, the system processor 310 or any component thereof. The biosensor electrode terminal 322 is operable to operatively couple the electrode array 320 to the biological surface 250. In some implementations, the biosensor electrode terminal 322 includes one or more of the electrode layer 210, the electrode material 212, and the biochemical sensor material 214 associated with, contained within, partially surrounded by, or the like, a corresponding fluid chamber 222. In some implementations, a single biosensor electrode terminal 322 corresponds to a single electrode of the electrochemical sensor 140. In some implementations, the electrochemical sensor 140 includes a plurality of biosensor electrode terminals 322. In some implementations, the electrochemical sensor 140 includes a plurality of biosensor electrode terminals 322 arranged in a grid or like configuration.

The iontophoresis inducer 330 is operable to control, generate, define, or the like, one or more signals, pulses, or the like, of electrical energy applied to the biological surface according to one or more electrical output patterns. In some implementations, the iontophoresis inducer 330 is operable to apply electrical energy to the biological surface in accordance with an iontophoresis process. In some implementations, the stimulation module 130 includes the iontophoresis inducer 330. In some implementations, the iontophoresis inducer 330 is operable to induce a biological reaction from the biological surface in accordance with the operation of the stimulation module 130. In some implementations, the iontophoresis inducer 330 includes one or more electrical, electronic, and logical devices. In some implementations, the iontophoresis inducer 330 includes one or more integrated circuits, transistors, transistor arrays, or the like. The iontophoresis electrode terminal 332 is operable to apply one or more signals, pulses, or the like, of electrical energy to the biological surface according to one or more electrical output patterns in response to signals, instructions, or the like received from the iontophoresis inducer 330. In some implementations, the stimulation module 130 includes the iontophoresis electrode terminal 332. In some implementations, the iontophoresis electrode terminal include at least one conductive electrode material, and a conductive lead, wire, connector, or the like.

The communication interface 340 is operable to communicatively couple at least the system processor 310 to at least one external device. In some implementations, the communication interface 114 includes one or more wired interface devices, channels, and the like. In some implementations, the communication interface includes, is operably coupled to, or is operably couplable to an I2C, UART, or like communication interface by one or more external devices, systems, or the like. In some implementations, the communication interface includes a network or an Internet communication interface or is operably couplable to an Internet communication interface by one or more external devices, systems, or the like. The display device 120 is operable to visually communicate one or more electrical responses received at the electrode 130. In some implementations, the display device is operably coupled to at least the system processor 310.

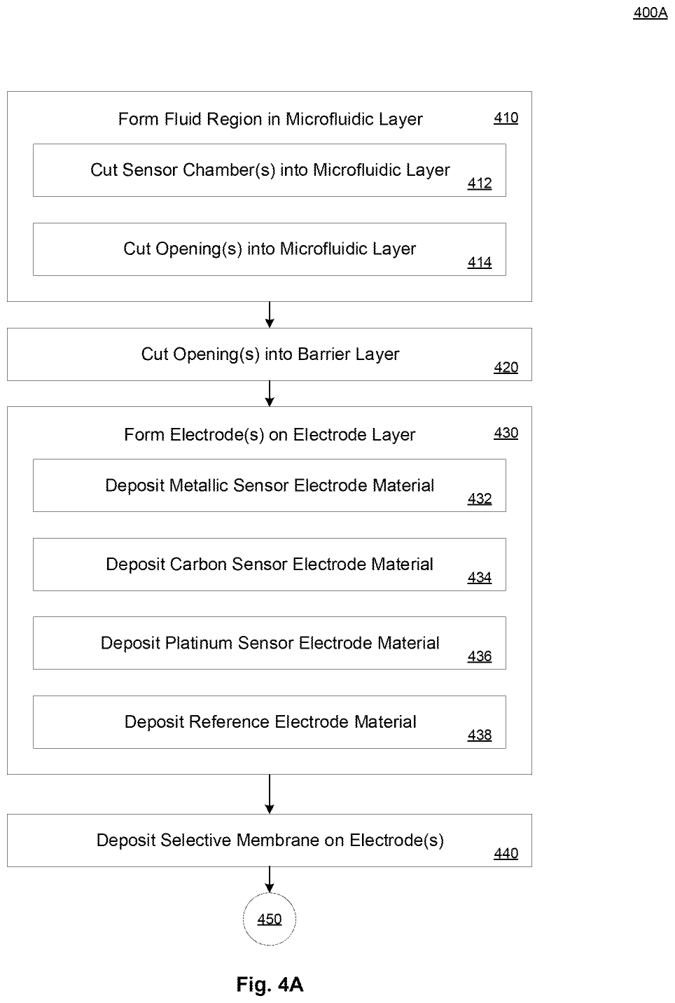

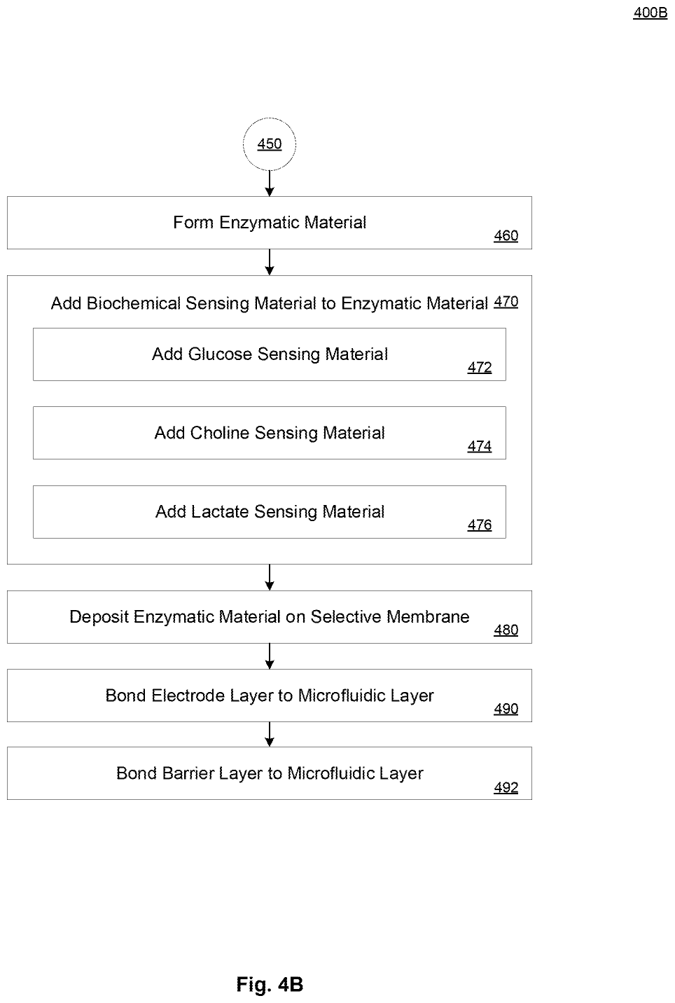

FIG. 4A illustrates an example method of manufacturing an example electrochemical sensor in accordance with present implementations. In some implementations, at least one of the example device 100A and 100B is formed by method 400A according to present implementations. In some implementations, the method 400A begins at step 410.

At step 410, the example system forms a fluid region in a microfluidic layer. In some implementations, the example system forms the fluid region by removing at least a portion of the microfluidic layer 220. In some implementations, the example system removes one or more portions of the microfluidic layer 220 by forming at least one opening, cavity, or the like, in a depth direction of the microfluidic layer 220 perpendicular to a plane thereof. In some implementations, the example system removes all material within a predetermined region of the microfluidic layer 220. In some implementations, the example system forms at least one fluid chamber 222 in accordance with at least one pattern or the like. In some implementations, step 410 includes at least one of steps 412 and 414. At step 412, the example system cuts a sensor chamber into the microfluidic layer. In some implementations, the example system removes a portion of the microfluidic layer by etching a sensor chamber into the microfluidic chamber 220. In some implementations, the etching includes etching by one or more lasers in a cutting or like action. In some implementations, the sensor chamber corresponds to at least one fluid chamber 222. At step 414, the example system cuts an opening into the microfluidic layer. The method 400A then continues to step 420.

At step 420, the example system cuts an opening into a barrier layer. In some implementations, the example system etches the opening into the barrier layer 240 by removing at least a portion of the barrier layer 240. In some implementations, the example system removes one or more portions of the barrier layer 240 by forming at least one opening, cavity, or the like, in a depth direction of the barrier layer 240 perpendicular to a plane thereof. In some implementations, the example system removes one or more portions of the barrier layer 240 correspondingly to step 410. In some implementations, the example system removes all material within a predetermined region of the barrier layer 240. In some implementations, the example system forms at least one opening into the barrier layer 240 in alignment with or corresponding to the fluid chamber 222. The method 400A then continues to step 430.

At step 430, the example system forms an electrode on an electrode layer. In some implementations, step 430 includes at least one of steps 432, 434, 436 and 438. At step 432, the example system deposits metallic sensor electrode material directly or indirectly on the electrode layer. In some implementations, the exemplary system deposits a 200 nm thick layer of Au onto the electrode layer 210 to form the base electrode layer 212. In some implementations, the exemplary system optionally deposits a Cr layer on the electrode layer before depositing Au on the electrode layer 210. At step 434, the example system deposits carbon sensor electrode material directly or indirectly on the electrode layer 210. In some implementations, a multi-walled carbon nanotube solution is prepared by mixing modified multi-walled carbon nanotubes (MWCNT) at a concentration of 2 mg per mL, with a 5 wt. % Nafion® solution. Further, in some implementations, the MWCNT solution is then treated by ultrasonic agitation over 30 min to form a viscous solution of carbon nanotubes. In some implementations, 3.2 μL of the MWCNT solution is drop-cast onto the Au electrode and dried in the ambient environment to form the carbon nanotube layer 214.

At step 436, the example system deposits platinum sensor electrode material. In some implementations, the electrode material includes platinum nanoparticles (PtNP). In some implementations, PtNP is electrochemically deposited onto the MWCNT deposited on the carbon nanotube layer 214 by chronoamperometry at −0.1 V (versus Ag/AgCl) for 10 min in a fresh Pt-solution. In some implementations, the fresh Pt-solution includes 2.5 mM H₂PtCl₆ and 1.5 mM formic acid. By electrochemically depositing PtNP onto the Au electrode, H₂O₂ can be effectively oxidized at 0.5 V (versus Ag/AgCl). At step 438, the example system deposits reference electrode material. In some implementations, a reference electrode is formed by capping one or more electrodes with a reference electrode material. In some implementations, the reference electrode material includes silver (Ag) or silver chloride (AgCl) ink. The reference electrode was fabricated by In some implementations, forming the reference electrode includes depositing 3.2 μL of Ag/AgCl ink on the Au electrode, followed by drying at 70° C. for 20 min. In some implementations, the method 400A then continues to step 440.

At step 440, the example system deposits a selective membrane on the biochemical sensor electrode. In some implementations, a poly-m-phenylenediamine (PPD) layer is electrochemically deposited onto the platinum layer 216 by applying 0.85 V (versus Ag/AgCl) for 120 s in a fresh PBS solution. In some implementations, the PBS solution is a 1×PBS solution containing 5 mM m-phenylenediamine. In some implementations, the PPD layer is electrochemically deposited onto the platinum layer 216 following a method corresponding to PtNP and MWCNT deposition. The method 400A then continues to step 450.

FIG. 4B illustrates an example method of manufacturing an example electrochemical sensor further to the example method of FIG. 4A. In some implementations, at least one of the example device 100A and 100B is formed by method 400B according to present implementations. In some implementations, the method 400B begins at step 450. The method 400B then continues to step 460.

At step 460, the example system forms an enzymatic material. In some implementations, the biochemical sensor electrode is functionalized with an enzymatic layer. In some implementations, the enzymatic material is or includes a bovine serum albumin (BSA) stabilizer. In some implementations, BSA stabilizer solution is prepared by adding 0.8% (v/v) of 25 wt. % glutaraldehyde solution into a fresh PBS solution containing 10 mg mL-1 BSA. The method 400B then continues to step 470.

At step 470, the example system adds a biochemical sensing material to the enzymatic material. In some implementations, step 470 includes at least one of steps 472, 474, 476 and 478. At step 472, the example system adds a glucose sensing material to the enzymatic material. In some implementations, to develop a glucose sensor, the BSA stabilizer solution is mixed with a glucose oxidase solution at a concentration of 50 mg per mL in PBS at pH 7.2. In some implementations, the BSA stabilizer solution and the glucose oxidase are mixed at a ratio of 1:1. In some implementations, the mixture is then drop cast onto the selective membrane 218. In some implementations, the mixture is drop cast in an amount of 3.2 μL.

At step 454, the example system adds a choline sensing material to the enzymatic material. In some implementations, to develop a choline sensor, the BSA stabilizer solution is mixed with a choline oxidase solution at a concentration of 10 mg per mL in PBS at pH 7.2. In some implementations, the BSA stabilizer solution and the choline oxidase are mixed at a ratio of 1:1. In some implementations, the mixture is then drop cast onto the selective membrane 218. In some implementations, the mixture is drop cast in an amount of 3.2 μL.

At step 456, the example system adds a lactate sensing material to the enzymatic material. In some implementations, to create the lactate sensor, the BSA stabilizer solution is mixed with a lactate oxidase solution at a concentration of 50 mg per mL in PBS at pH 7.2. In some implementations, the BSA stabilizer solution and the lactate oxidase are mixed at a ratio of 1:1. In some implementations, the mixture is then drop cast onto the selective membrane 218. In some implementations, the mixture is drop cast in an amount of 3.2 μL. In some implementations, the mixture is allowed to dry, and applied with a dip-coating of 3 wt. % PVC solution to form a lactate diffusion limiting layer. The method 400B then continues to step 480. At step 480, the example system deposits the enzymatic material on the selective membrane. In some implementations, the enzymatic material includes one of a glucose sensing mixture, a choline sensing mixture, and a lactate sensing mixture. The method 400B then continues to step 490.

At step 490, the example system bonds the electrode layer to the microfluidic layer. In some implementations, at least one of the electrode layer 210 and the microfluidic layer 220 includes an adhesive material disposed on at least one planar surface thereof. In some implementations, the electrode layer 210 is bonded to the microfluidic layer 220 with the electrodes of the electrode layer 210 in alignment with the fluid chamber 222 of the microfluidic layer 220 in one or more planar directions of both layers 210 and 220. In some implementations, a planar surface of the electrode layer 210 on which the electrodes are formed is bonded to a planar surface of the microfluidic layer 220. Thus, in some implementations, the bonding of the electrode layer 210 and the microfluidic layer 220 results in the formation of at least one electrode chamber having at least one electrode at least partially surrounded at its sides by walls of the fluid chamber 222. The method 400B then continues to step 492.

At step 492, the example system bonds the barrier layer to the microfluidic layer. In some implementations, at least one of the barrier layer 240 and the microfluidic layer 220 includes an adhesive material disposed on at least one planar surface thereof. In some implementations, the barrier layer 240 is bonded to the microfluidic layer 220 with one or more openings of the barrier layer 240 in alignment with the fluid chamber 222 of the microfluidic layer 220 in one or more planar directions of both layers 210 and 220. Thus, in some implementations, the bonding of the barrier layer 240 and the microfluidic layer 220 results in the formation of at least one electrode chamber having at least one electrode at least partially enclosed at its sides by walls of the fluid chamber 222, and at a side opposite to the electrode, by the barrier layer 240. In some implementations, the method 400B ends at step 492.

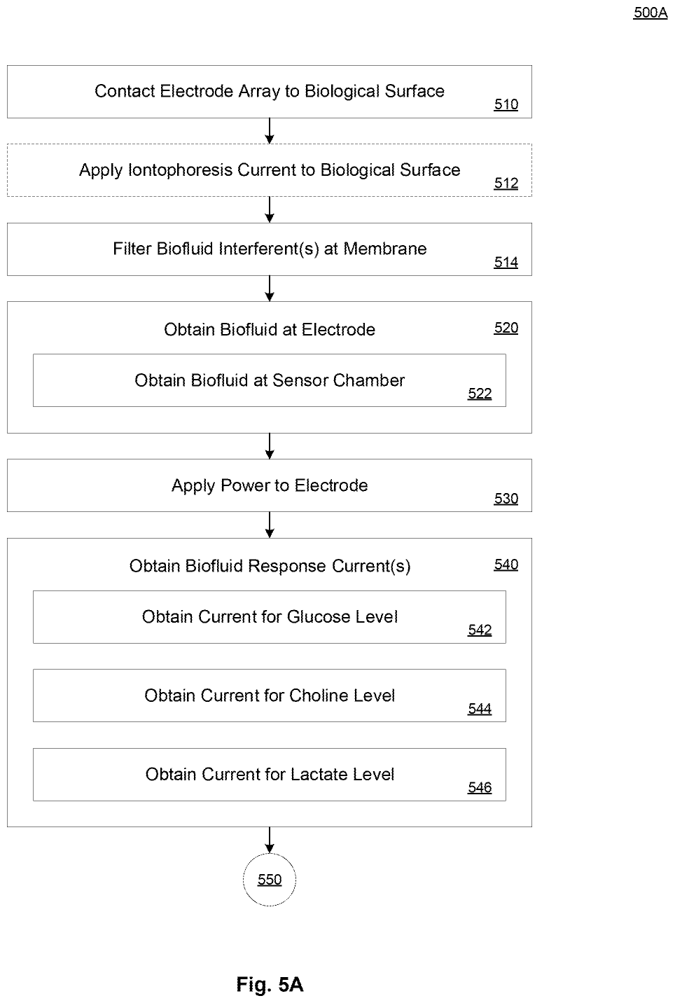

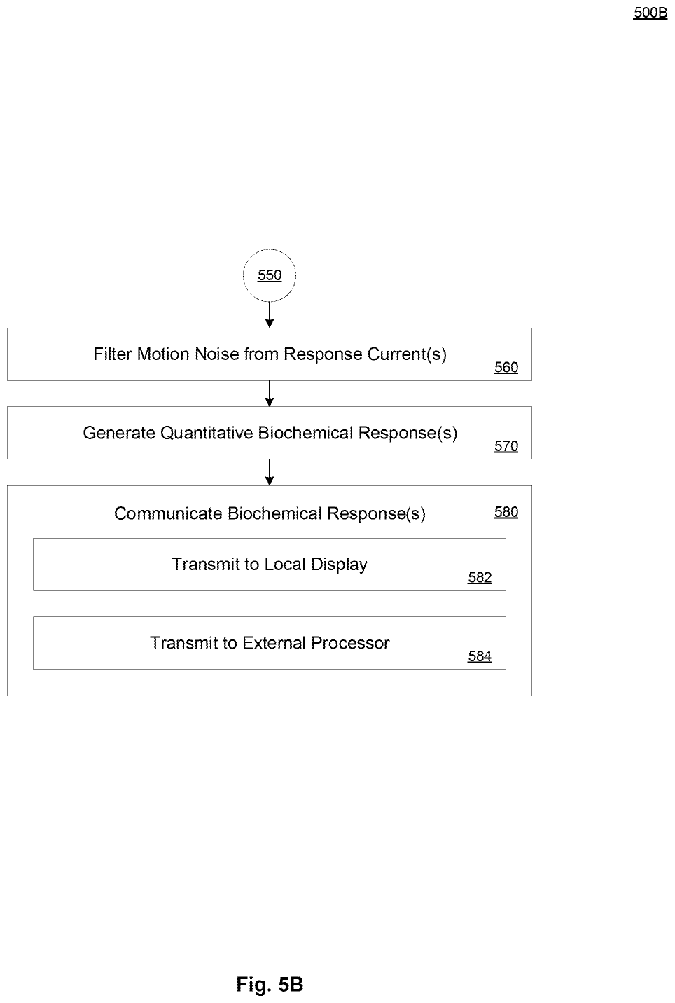

FIG. 5A illustrates an example method of electrically sensing a biochemical in accordance with present implementations. In some implementations, at least one of the example device 100A and 100B performs method 500A according to present implementations. In some implementations, the method 500A begins at step 510.

At step 510, the example system contacts an electrode to a biological surface. In some implementations, the example system contacts the electrode 130 to the biological surface 250 by the adhesive layer 240. The method 500A then continues to step 512.

At step 512, the example system applies an iontophoresis current to the biological surface. In some implementations, iontophoresis inducer 330 applies a current independent of a user's skin type or skin impedance. In some implementations, the applied current is limited through the current limiting circuits implemented on board and constantly monitored by the system processor 310 to apply a minimal current. In some implementations, the minimal current is set by the user in a comfortable range of 0.1-1.5 mA. It is to be understood that the example system can optionally apply the iontophoresis current. It is to be understood that the example system can optionally apply the iontophoresis current based on control of one or more of the system processor 310, the iontophoresis inducer 330, the communication interface 340, and the stimulation module 130. In some implementations, the applied iontophoresis current increases an amount or rate of secretion of a biofluid from the biological surface 250. In some implementations, the iontophoresis current is applied constantly or according to a predetermined pattern to induce sweat or to induce a particular amount or rate of sweat secretion from the biological surface 250. The method 500A then continues to step 514.

At step 514, the example system filters one or more biofluid interferents at the selective membrane. As one example, interferents include glucose, lactate, creatinine, choline, potassium chloride, sodium chloride, uric acid, ascorbic acid, pilocarpine, aspirin, metformin, and albumin. In some implementations, interfering species include, but are not limited to, small molecules, ionic species, electroactive species, a sweat agonist, xenobiotic drugs, and protein. In some implementations, glucose, lactate, and choline sensors show negligible current responses towards one or more interfering species, are responsive upon introduction of the sensor-specific target molecules with significant stepwise current increases. The method 500A then continues to step 520.

At step 520, the example system obtains a biofluid at the biochemical sensor electrode. In some implementations, the biofluid secreted from the biological surface 250 travels to the electrode by one or more of the microfluidic layer 220 and the barrier layer 240. In some implementations, the biofluid travels into an opening of the barrier layer 240 from the biological surface 250, then into the opening of the microfluidic layer 220. In some implementations, step 520 includes step 522. At step 522, the example system obtains the biofluid at a sensor chamber. In some implementations, the biofluid travels into the fluid chamber 222. The method 500A then continues to step 530.

At step 530, the example system applies power to the electrode array. In some implementations, the example system applies a constant 0.5 V current to electrodes of the electrode array, including biochemically responsive and reference biosensor electrode terminals 332. In some implementations, the example system applies the iontophoresis current to the biological surface 250 by the iontophoresis electrode terminal 332 concurrently with applying power to the electrode terminal 332 of the electrode array. The method 500A then continues to step 540.

At step 540, the example system obtains a biofluid response current. In some implementations, a peak supply current level is 100 mA. In some implementations, detection of H₂O₂ occurs with a sensitivity of 558±25 μA/mM/cm2 in a range from 50 μM to 1.1 mM. In some implementations, the biochemical sensor electrode advantageously shows minimum sensitivity drift from interference from variation in ionic species concentration levels in an example biofluid matrix, including but not limited to human sweat. In some implementations, a biochemical sensor electrode including a platinum layer 216 is minimally influenced by addition of K+. As one example, an example sensor can possess a ˜1% signal difference for 6 mM K+. Given the variation of K+ in biofluid (e.g., sweat) samples, a biochemical sensor electrode in accordance with present implementations advantageously gives a reliable reading in the presence of naturally occurring interferents. In some implementations, a biochemical sensor electrode in accordance with present implementations can advantageously exhibits more than eight times the sensitivity of mediator-based system, to reach 399±50 μA/mM/cm2. In some implementations, step 540 includes at least one of steps 542, 544 and 546. At step 542, the example system obtains a response current associated with a glucose level at the electrode array. In some implementations, a glucose response sensitivity is 59±12 μA/mM/cm2 for glucose concentrations from 0 to 300 μM. At step 544, the example system obtains a response current associated with a choline level at the electrode array. In some implementations, a choline response sensitivity is 68±5 μA/mM/cm2 for choline concentrations from 0 to 300 μM. At step 546, the example system obtains a response current associated with a lactate level at the electrode array. In some implementations, a lactate response sensitivity is 0.79±0.18 μA/mM/cm2 for lactate concentrations from 5 to 20 mM. In some implementations, the method 500A then continues to step 550.

FIG. 5B illustrates an example method of electrically sensing a biochemical further to the example method of FIG. 5A. In some implementations, at least one of the example device 100A and 100B performs method 500B according to present implementations. In some implementations, the method 500B begins at step 550. The method 500B then continues to step 560.

At step 560, the example system filters motion noise from the response current. In some implementations, the system processor 310 filters motion noise from the response current. In some implementations, motion noise includes variations of a response current generated in response to physical movement of an electrode relative to a biological surface. As one example, physical movement can include shaking, shifting, jostling, shaking, vibrating, or the like of a housing including a sensor device. In some implementations, physical movement of the housing 110 is generated in response to physical movement of a wearer of a device including the electrode array 320. In some implementations, the example system filters the motion noise by passing the response current through a low pass filter to remove one or more high frequency artifacts from the response current. The method 500B then continues to step 570.

At step 570, the example system generates a quantitative biochemical response. In some implementations, the example system generates the quantitative biochemical response by converting an analog response current to a digital response. In some implementations, the system processor generates the quantitative biochemical response by an analog-to-digital converter therein or therewith. The method 500B then continues to step 580.

At step 580, the example system communicates the biochemical response. In some implementations, step 580 includes at least one of steps 582 and 584. At step 582, the example system transmits the biochemical response to the local display. At step 584, the example system transmits the biochemical response to an external processor. In some implementations, the external processor includes a remote server, remote computer, remote database, and the like. In some implementations, the method 500B ends at step 580.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A method of manufacturing a biochemical sensor, the method comprising:
forming a fluid region in a microfluidic layer;
forming a reference electrode on a planar surface of an electrode layer;
forming a biochemical sensor electrode on the planar surface;
forming a selective membrane on the biochemical sensor electrode;
forming an enzymatic material including a biochemical sensing material on the selective membrane; and
bonding the electrode layer to the microfluidic layer.

2. The method of claim 1, further comprising:
mixing the biochemical sensing material with a stabilizer solution to form the enzymatic material including the biochemical sensing material.

3. The method of claim 1, wherein the stabilizer solution comprises a bovine serum albumin stabilizer solution.

4. The method of claim 1, wherein the forming the fluid region comprises removing a portion of the microfluidic layer to form a sensor chamber.

5. The method of claim 1, wherein the forming the biochemical sensor electrode comprises depositing a gold electrode material on the electrode layer.

6. The method of claim 5, wherein the forming the biochemical sensor electrode further comprises depositing a carbon nanotube electrode material on the gold electrode material.

7. The method of claim 6, wherein the forming the biochemical sensor electrode further comprises depositing a platinum electrode material on the carbon nanotube electrode material.

8. The method of claim 1, wherein the forming the reference electrode further comprises depositing a gold electrode material on the electrode layer, and depositing a silver chloride electrode material on the gold electrode material.

9. The method of claim 1, wherein the biochemical sensing material comprises a glucose sensing material.

10. The method of claim 1, wherein the biochemical sensing material comprises a choline sensing material.

11. The method of claim 1, wherein the biochemical sensing material comprises a lactate sensing material.

12. A device comprising:
a reference electrode disposed on a planar surface of an electrode layer;
a biochemical sensor electrode disposed on the planar surface;
a selective membrane disposed on the biochemical sensor electrode and impermeable to at least one biochemical interferent; and
an enzymatic layer disposed on the selective membrane and electrically responsive to a biochemical.

13. The device of claim 12, further comprising:
a carbon nanotube electrode material disposed on the biochemical sensor electrode.

14. The device of claim 13, further comprising:
a platinum electrode material disposed on the carbon nanotube electrode material.

15. The device of claim 14, further comprising:
a selective membrane disposed on the platinum electrode.

16. The device of claim 12, wherein the enzymatic layer includes glucose oxide and a stabilizer material, and is electrically responsive to at least indirect contact with glucose.

17. The device of claim 12, wherein the enzymatic layer includes choline oxide and a stabilizer material, and is electrically responsive to at least indirect contact with choline.

18. The device of claim 12, wherein the enzymatic layer includes lactate oxide and a stabilizer material, and is electrically responsive to at least indirect contact with lactate.

19. The device of claim 12, further comprising:
a microfluidic layer disposed on the electrode layer and comprising a sensor chamber region disposed at least partially surrounding at least one of the reference electrode and the biochemical sensor electrode.

20. A method of electrically detecting a biochemical, the method comprising:
contacting a biochemical sensor electrode to a biological surface;
obtaining a biofluid at the biochemical sensor electrode from the biological surface;
filtering an interferent at a selective membrane disposed between the biochemical sensor electrode and the biological surface;
obtaining a response current associated with the biofluid at the biochemical sensor electrode; and
generating a quantitative biochemical response based at least partially on the response current.
